(19) United States
(12) Patent Application Publication
(10) Pub. No.: US 2018/0221316 A1
(43) Pub. Date: Aug. 9, 2018

(54) METHODS AND MATERIALS FOR REDUCING DAMAGE TO TRANSPLANTED TISSUE

(71) Applicant: THE UNIVERSITY OF PÉCS (PÉCSI TUDOMÁNYEGYETEM), Pecs (HU)

(72) Inventor: Agnes NAGY, Pecs (HU)

(21) Appl. No.: 15/748,439

(22) PCT Filed: Aug. 3, 2016
(86) PCT No.: PCT/IB2016/054699

§ 371 Date: Jan. 29, 2018

Related U.S. Application Data
(60) Provisional application No. 62/202,052, filed on Aug. 6, 2015.

(51) Int. Cl. A61K 31/19; A61K 9/00; A01N 1/02

(52) U.S. Cl. CPC A61K 31/19; A01N 1/0226; A61K 9/0019

(57) ABSTRACT

This document provides methods and materials for reducing the degree of damage to tissue transplanted into a mammal following transplantation. For example, methods and materials for using abscisic acid to reduce tissue damage to vascularized tissue transplanted into a mammal following reperfusion of that vascularized tissue are provided.

### CROSS REFERENCE TO RELATED APPLICATIONS

This application claims the benefit of U.S. Provisional Ser. No. ______ filed ______. This disclosure of the prior application is considered part of (and is incorporated by reference in) the disclosure of this application.

### BACKGROUND

### 1. Technical Field

This document relates to methods and materials for reducing the degree of damage to tissue transplanted into a mammal following transplantation. For example, this document relates to using abscisic acid to reduce tissue damage to vascularized tissue transplanted into a mammal following reperfusion of that vascularized tissue.

### 2. Background Information

Organ transplantation generally involves obtaining an organ from one mammal and implanting it into another mammal. In these cases, there is a risk that the transplanted organ will be damaged from the reperfusion of the organ.

### SUMMARY

This document provides methods and materials for reducing the degree of damage to transplanted tissue (e.g., vascularized transplanted tissue) that is induced by the reperfusion of that transplanted tissue. For example, this document provides methods and materials for administering abscisic acid to reduce the degree of tissue injury experienced by transplanted tissue that is caused by reperfusion of the transplanted tissue during a transplantation procedure. Abscisic acid (ABA) is also known as (2Z,4E)-5-[(1S)-1-hydroxy-2,6,6-trimethyl-4-oxocyclohex-2-en-1-yl]-3-methylpenta-2,4-dienoic acid and (2Z,4E)-(S)-5-(1-hydroxy-2,6,6-trimethyl-4-oxo-2-cyclohexen-1-yl)-3-methyl-2,4-pentanedienoic acid.

As described herein, mammals undergoing a transplantation procedure (e.g., a liver, kidney, or heart transplantation procedure) can be administered ABA in a manner that reduces the severity of tissue or cell injury following reperfusion of the transplanted tissue. In some cases, ABA can be administered to a mammal before reperfusion of the transplanted tissue. For example, a human patient undergoing an organ transplantation procedure (e.g., a kidney transplantation procedure) can be administered ABA about 2 minutes to 60 minutes prior to reperfusion of the transplanted organ to reduce the severity of tissue or cell injury caused by the reperfusion.

In some cases, the transplantation tissue can be exposed to ABA prior to being transplanted into a recipient. For example, the donor can be administered ABA prior to (e.g., 2 minutes to 24 hours prior to) removal of the transplantation tissue. In such cases, the removed transplantation tissue can be exposed to additional ABA (e.g., a solution containing ABA) or can be stored without being exposed to additional ABA. In some cases, transplantation tissue (e.g., a liver, kidney, or heart) can be removed from a donor without having been exposed to exogenous ABA. In such cases, the removed transplantation tissue can be exposed to ABA prior to being transplanted into a recipient. For example, the removed transplantation tissue can be maintained in a solution containing ABA until transplanted into the recipient.

The use of ABA as described herein can help reduce damage to transplanted tissue (e.g., vascularized transplantation tissue) following reperfusion of the transplanted tissue. For example, administering ABA to a human recipient undergoing an organ transplantation can help reduce the level of damage to the transplanted tissue's architecture such that minimal extracellular oedema (e.g., minimal fiber separation) and a near complete absence of intercellular vacuolization occurs following reperfusion of the transplanted tissue as compared to that which occurs in comparable transplantation tissue when ABA is not used.

In general, one aspect of this document features a method for reducing damage to transplantation tissue induced by reperfusion of the transplantation tissue in a mammalian recipient. The method comprises, or consists essentially of, administering a composition comprising abscisic acid to the mammalian recipient, wherein the level of damage of the transplantation tissue induced by reperfusion of the transplantation tissue in the mammalian recipient is reduced as compared to the level of damage of comparable transplantation tissue transplanted into a comparable mammalian recipient not administered the composition. The mammalian recipient can be a human. The transplantation tissue can be a kidney, liver, heart, lung, pancreas, thymus, or cardiac valve. The composition can be administered to the mammalian recipient during a transplantation procedure. The composition can be administered to the mammalian recipient at least 30 seconds before reperfusion of the transplantation tissue. The composition can comprise between 5 and 95 percent by weight the abscisic acid. The composition can comprise between 20 and 80 percent by weight the abscisic acid. The composition can comprise between 0.1 and 10 percent by weight the abscisic acid. The composition can comprise a saline solution. The administration can comprise an intravenous administration.

In another aspect, this document features a method for reducing damage to transplantation tissue induced by reperfusion of the transplantation tissue in a mammalian recipient. The method comprises, or consists essentially of, administering a composition comprising abscisic acid to a donor of the transplantation tissue, wherein the level of damage of the transplantation tissue induced by reperfusion of the transplantation tissue in the mammalian recipient is reduced as compared to the level of damage of comparable transplantation tissue obtained from a comparable donor not treated with the composition and implanted into a comparable mammalian recipient. The mammalian recipient can be a human. The donor can be a human. The transplantation tissue can be a kidney, liver, heart, lung, pancreas, thymus, or cardiac valve. The composition can be administered to the donor at least 12 hours before the transplantation tissue is removed from the donor. The composition can comprise between 5 and 95 percent by weight the abscisic acid. The composition can comprise between 20 and 80 percent by weight the abscisic acid. The composition can comprise between 0.1 and 10 percent by weight the abscisic acid. The composition can comprise a saline solution. The administration of the composition can comprise an intravenous administration. The method can comprise administering, to the mammalian recipient, a second composition comprising, or consisting essentially of, abscisic acid. The method can comprise administering a second composition comprising, or consisting essentially of, abscisic acid to the mammalian recipient at least 30 seconds before reperfusion of the transplantation tissue. The second composition can comprise between 5 and 95 percent by weight the abscisic acid. The second composition can comprise between 20 and 80 percent by weight the abscisic acid. The second composition can comprise between 0.1 and 10 percent by weight the abscisic acid. The second composition can comprise a saline solution. The administration of the second composition can comprise an intravenous administration.

In another aspect, this document features a method for reducing damage to transplantation tissue induced by reperfusion of the transplantation tissue in a mammalian recipient. The method comprises, or consists essentially of, (a) contacting transplantation tissue with a composition comprising abscisic acid prior to implanting the transplantation tissue into the mammalian recipient, (b) implanting the transplantation tissue into the mammalian recipient, and (c) reperfusing the transplantation tissue, wherein the level of damage of the transplantation tissue induced by reperfusion of the transplantation tissue in the mammalian recipient is reduced as compared to the level of damage of comparable transplantation tissue not contacted with the composition and transplanted into a comparable mammalian recipient. The mammalian recipient can be a human. The method of any one of claims 28-29, wherein the transplantation tissue can be a kidney, liver, heart, lung, pancreas, thymus, or cardiac valve. The method of any one of claims 28-30, wherein the composition can be contacted with the transplantation tissue at least 30 minutes prior to implanting the transplantation tissue into the mammalian recipient. The method of any one of claims 28-31, wherein the composition can comprise between 5 and 95 percent by weight the abscisic acid. The method of any one of claims 28-32, wherein the composition can comprise between 20 and 80 percent by weight the abscisic acid. The method of any one of claims 28-31, wherein the composition can comprise between 0.1 and 10 percent by weight the abscisic acid. The method of any one of claims 28-34, wherein the composition can comprise a saline solution. The method of any one of claims 28-35, wherein the method can comprise administering a composition comprising abscisic acid to the mammalian recipient. The method can comprise administering a composition comprising abscisic acid to the mammalian recipient at least 30 seconds before the reperfusing step. The method can comprise administering a composition comprising abscisic acid to a donor of the transplantation tissue prior to removal of the transplantation tissue from the donor. The method can comprise administering a composition comprising abscisic acid to a donor of the transplantation tissue at least 10 minutes prior to removal of the transplantation tissue from the donor.

In another aspect, this document features a composition comprising, or consisting essentially of, abscisic acid for use in the manufacture of a medicament for reducing damage to transplantation tissue induced by reperfusion of the transplantation tissue in a mammalian recipient.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and from the claims.

### DETAILED DESCRIPTION

This document provides methods and materials for reducing the degree of damage to transplanted tissue (e.g., vascularized transplanted tissue) that is induced by the reperfusion of that transplanted tissue. For example, this document provides methods and materials for using abscisic acid to reduce the degree of tissue injury experienced by transplanted tissue that is caused by reperfusion of the transplanted tissue during a transplantation procedure.

The methods and materials provided herein can be used with any appropriate transplant procedure to reduce the degree of damage induced by the reperfusion of the transplanted tissue. For example, the methods and materials provided herein can be used when transplanting liver, kidney, heart, lung, pancreas, thymus, or cardiac valve tissue or organs into a recipient. In some cases, the transplantation can be an auto-transplantation (e.g., transplantation of organs or tissues from one part of a recipient's body to another part of the same recipient's body), an allo-transplantation (e.g., transplantation of an organ or tissue from a donor of one species to a recipient of the same species), or a xeno-transplantation (e.g., transplantation of an organ or tissue from a donor of one species to a recipient of a different species). The recipients can be any appropriate recipients including, without limitation, mammals such as humans, monkeys, dogs, cats, members of a bovine species, horses, pigs, rats, and mice. Likewise, the donors can be any appropriate donors including, without limitation, mammals such as humans, monkeys, dogs, cats, members of a bovine species, horses, pigs, rats, and mice. In some cases, a donor of an organ or a tissue to be transplanted can be a living donor (e.g., a donor who is alive when the transplantation tissue is removed) or a deceased donor (e.g., a donor who is not alive when the transplantation tissue is removed).

At any point during a transplantation procedure, ABA can be administered to the recipient (e.g., a human recipient) to reduce the level of injury that normally occurs following reperfusion of the transplanted tissue. For example, after the transplantation procedure starts but before one or more blood vessels are occluded (e.g., about 10 seconds to about 120 minutes prior to occluding one or more blood vessels), ABA can be administered to the recipient. In some cases, ABA can be administered to the recipient during the surgical process from about 10 seconds to about 90 minutes (e.g., from about 10 seconds to about 60 minutes, from about 10 seconds to about 30 minutes, from about 10 seconds to about 15 minutes, from about 10 seconds to about 10 minutes, from about 10 seconds to about 5 minutes, from about 10 seconds to about 2 minutes, or from about 10 seconds to about 1 minute) prior to occluding one or more blood vessels.

In another example, ABA can be administered to the recipient after the transplantation procedure starts but before the transplanted tissue is reperfused (e.g., about 10 seconds to about 120 minutes prior to reperfusing the transplanted tissue). In some cases, ABA can be administered to the recipient during the surgical process from about 10 seconds to about 90 minutes (e.g., from about 10 seconds to about 60 minutes, from about 10 seconds to about 30 minutes, from about 10 seconds to about 15 minutes, from about 10 seconds to about 10 minutes, from about 10 seconds to about 5 minutes, from about 10 seconds to about 2 minutes, or from about 10 seconds to about 1 minute) prior to reperfusing the transplanted tissue.

In some cases, ABA can be administered to a transplantation recipient during the transplantation procedure while one or more blood vessels are being surgically occluded. For example, ABA can be administered to a transplantation recipient as a single administration (e.g., an injection) while one or more blood vessels are being surgically occluded. In another embodiment, a transplantation recipient can be provided an IV solution containing ABA that delivers ABA to the transplantation recipient the entire time one or more blood vessels are being surgically occluded. In some cases, ABA can be administered during the transplantation procedure at the same time that reperfusion of the transplanted tissue is initiated.

In some cases, ABA can be administered to a transplantation procedure during the transplantation procedure but after removal of one or more surgically applied occlusions. For example, ABA can be administered during the transplantation procedure from about 1 second to about 90 minutes (e.g., from about 1 second to about 60 minutes, from about 1 second to about 30 minutes, from about 1 second to about 15 minutes, from about 1 second to about 10 minutes, from about 1 second to about 5 minutes, from about 1 second to about 2 minutes, or from about 1 second to about 1 minute) following removal of one or more surgically applied occlusions.

In some cases, ABA can be administered during the entire transplantation procedure. For example, a transplantation recipient can be provided an IV solution containing ABA that delivers ABA to the transplantation recipient the entire time during a transplantation procedure.

In some cases, ABA can be administered to a recipient (e.g., a human recipient) prior to initiation of the transplantation procedure to reduce the level of injury that normally occurs following reperfusion of the transplanted tissue. For example, ABA can be administered to a recipient one to six times a day for at least one, two, three, four, five, or more days before the transplantation procedure. In some cases, ABA can be administered to a recipient at least daily for one to four weeks prior to the transplantation procedure. In some cases, ABA can be administered both prior to the transplantation procedure and during the transplantation procedure.

In some cases, ABA can be administered before beginning a transplantation procedure. For example, ABA can be administered to a transplantation recipient from about 1 minute to about 48 hours before the transplantation procedure begins (e.g., from about 5 minutes to about 48 hours, from about 15 minutes to about 48 hours, from about 30 minutes to about 48 hours, from about 1 hour to about 48 hours, from about 2 hours to about 48 hours, from about 3 hours to about 48 hours, from about 4 hours to about 48 hours, from about 6 hours to about 48 hours, from about 8 hours to about 48 hours, from about 10 hours to about 48 hours, from about 12 hours to about 48 hours, from about 24 hours to about 48 hours, from about 1 minute to about 36 hours, from about 1 minute to about 24 hours, from about 1 minute to about 12 hours, from about 1 minute to about 8 hours, from about 1 minute to about 6 hours, or from about 1 minute to about 2 hours before the transplantation procedure begins) to reduce the severity of tissue damage caused by reperfusion.

In some cases, ABA can be administered before beginning a transplantation procedure and during the entire transplantation procedure. For example, a transplantation recipient can be administered ABA (e.g., by injection) prior to starting a transplantation procedure and then provided an IV solution containing ABA that delivers ABA to the recipient the entire time during the transplantation procedure.

In some cases, when ABA is administered before the transplantation procedure, during the transplantation procedure, or both before and during the transplantation procedure, the recipient can be released with no further administration of ABA to the recipient. For example, a human transplantation recipient can be administered ABA during the transplantation procedure with no further ABA being administered to that recipient once the transplantation procedure is completed. In some cases, a transplantation recipient can be administered ABA during the transplantation procedure and not for a period of at least one, two, three, four, five, or more days, weeks, or months after completion of the transplantation procedure.

In some cases, ABA can be administered to a transplantation recipient after the transplantation procedure is completed. For example, ABA can be administered to a recipient one to six times a day for at least one, two, three, four, five, or more days after the transplantation procedure completed. In some cases, ABA can be administered to a recipient at least daily for one to four weeks after the transplantation procedure is completed. In some cases, ABA can be administered to a transplantation recipient from about 1 minute to about 1 week after the transplantation procedure is completed (e.g., from about 5 minutes to about 1 week, from about 15 minutes to about 1 week, from about 30 minutes to about 1 week, from about 1 hour to about v, from about 2 hours to about 1 week, from about 3 hours to about 1 week, from about 4 hours to about 1 week, from about 6 hours to about 1 week, from about 8 hours to about 1 week, from about 10 hours to about 1 week, from about 12 hours to about 1 week, from about 24 hours to about 1 week, from about 1 minute to about 3 days, from about 1 minute to about 48 hours, from about 1 minute to about 24 hours, from about 1 minute to about 12 hours, from about 1 minute to about 8 hours, from about 1 minute to about 6 hours, from about 1 day to about 5 days, or from about 2 days to about 4 days after the transplantation procedure is completed) to reduce the severity of damage to the transplanted tissue by reperfusion.

In some cases, ABA can be administered (a) both prior to the transplantation procedure and after the transplantation procedure, (b) both during to the transplantation procedure and after the transplantation procedure, or (c) prior to the transplantation procedure, during the transplantation procedure, and after the transplantation procedure.

In some cases, the transplantation tissue can be exposed to ABA prior to being transplanted into a recipient. For example, the donor can be administered ABA prior to removal of the transplantation tissue. In some cases, ABA can be administered to a donor (e.g., a human transplantation donor) one to six times a day for at least one, two, three, four, five, or more days before removal of the transplantation tissue. In some cases, ABA can be administered to a donor at least daily for one to four weeks before the transplantation tissue is removed from the donor. In some cases, ABA can be administered to a transplantation donor from about 1 minute to about 1 week before the transplantation tissue is removed (e.g., from about 5 minutes to about 1 week, from about 15 minutes to about 1 week, from about 30 minutes to about 1 week, from about 1 hour to about v, from about 2 hours to about 1 week, from about 3 hours to about 1 week, from about 4 hours to about 1 week, from about 6 hours to about 1 week, from about 8 hours to about 1 week, from about 10 hours to about 1 week, from about 12 hours to about 1 week, from about 24 hours to about 1 week, from about 1 minute to about 3 days, from about 1 minute to about 48 hours, from about 1 minute to about 24 hours, from about 1 minute to about 12 hours, from about 1 minute to about 8 hours, from about 1 minute to about 6 hours, from about 1 day to about 5 days, or from about 2 days to about 4 days before the transplantation tissue is removed) to reduce the severity of damage to the transplanted tissue once reperfused within the recipient.

In some cases, the removed transplantation tissue that was exposed to ABA through the administration of ABA to the donor prior to removal of the transplantation tissue can be exposed to additional ABA (e.g., a solution containing ABA). For example, transplantation tissue exposed to ABA prior to removal from a donor can be placed into a solution containing ABA once removed from the donor. In some cases, this removed transplantation tissue can remain in the solution containing ABA until it is transplanted into a recipient. In some cases, removed transplantation tissue that was exposed to ABA through the administration of ABA to the donor prior to removal of the transplantation tissue can be maintained without being exposed to additional ABA.

In some cases, transplantation tissue can be removed from a donor that was not administered ABA prior to removal of the transplantation tissue. For example, removed transplantation tissue that was not exposed to exogenous ABA through the administration of ABA to the donor prior to removal of the transplantation tissue can be exposed to ABA (e.g., a solution containing ABA) for the first time by placing the removed transplantation tissue into a solution containing ABA. In some cases, the removed transplantation tissue can be maintained in a solution containing ABA until transplanted into the recipient.

As described herein, a recipient can be administered ABA before, during, or after a transplantation procedure, a donor can be administered ABA prior to removal of transplantation tissue, or transplantation tissue itself can be exposed directly to ABA prior to being transplanted into a recipient. In addition, any combination of these treatments can be performed. For example, a recipient can be administered ABA before, during, and after a transplantation procedure, a donor can be administered ABA prior to removal of transplantation tissue, and transplantation tissue itself can be exposed directly to ABA prior to being transplanted into a recipient. As another example, a recipient can be administered ABA before and during, but not after, a transplantation procedure, a donor can be administered ABA prior to removal of transplantation tissue, and transplantation tissue itself can be exposed directly to ABA prior to being transplanted into a recipient. As yet another example, a recipient can be administered ABA before and during, but not after, a transplantation procedure and a donor can be administered ABA prior to removal of transplantation tissue, but the transplantation tissue itself can be transplanted into the recipient without being exposed directly to ABA prior to being transplanted into the recipient.

In some cases, ABA (or a composition that includes ABA) can be administered to a transplantation recipient and/or a donor and/or can be used to treat transplantation tissue itself as described herein to reduce (e.g., reduce by at least 5, 10, 15, 20, 25, 30, 35, 40, 45, 50, 55, 60, 65, 70, 75, 80, 85, 90, 95 or 100 percent) the level of damage to the transplanted tissue following reperfusion of the transplanted tissue as compared to the level of damage observed when ABA is not administered or used. For example, the level of damage exhibited by transplanted tissue in a recipient administered ABA as described herein can be at least 5, 10, 15, 20, 25, 30, 35, 40, 45, 50, 55, 60, 65, 70, 75, 80, 85, 90, 95 or 100 percent less than that level exhibited by transplanted tissue in a comparable recipient not administered ABA.

Any appropriate amount of ABA can be administered to a mammal (e.g., transplantation donor and/or recipient) as described herein. In some cases, an effective amount of ABA can be administered to a transplantation donor and/or recipient to reduce the severity of damage induced by reperfusion of the transplanted tissue when implanted into the recipient. The term “effective” as used herein refers to any amount that induces a desired reduction in the level of damage induced by reperfusion of transplanted tissue within a recipient, while not inducing significant toxicity in the recipient and/or donor. Such an amount of ABA can be determined using animal models of transplantation and varying doses of ABA. The level of toxicity, if any, can be determined by assessing a donor's or recipient's clinical signs and symptoms before and after administering a known amount of ABA. It is noted that the effective amount of ABA administered to a donor and/or recipient can be adjusted according to a desired outcome as well as the donor's and/or recipient's response and level of toxicity. Significant toxicity can vary for each particular mammal.

In some cases, ABA can be administered to a donor and/or recipient in an amount that results in an ABA concentration within the donor's or recipient's blood that ranges from about 1 μM to about 15 mM (e.g., from about 1 μM to about 10 mM, from about 1 μM to about 5 mM, from about 1 μM to about 1 mM, from about 1 μM to about 0.9 mM, from about 1 μM to about 0.8 mM, from about 1 μM to about 0.7 mM, from about 1 μM to about 0.6 mM, from about 1 μM to about 500 μM, from about 1 μM to about 400 μM, from about 1 μM to about 300 μM, from about 1 μM to about 200 μM, from about 1 μM to about 100 μM, from about 1 μM to about 50 μM, from about 1 μM to about 10 μM, from about 5 μM to about 10 mM, from about 10 μM to about 10 mM, from about 25 μM to about 10 mM, from about 50 μM to about 10 mM, from about 75 μM to about 10 mM, from about 100 μM to about 10 mM, from about 250 μM to about 10 mM, from about 500 μM to about 10 mM, from about 750 μM to about 10 mM, from about 1 mM to about 10 mM, from about 10 μM to about 1 mM, from about 20 μM to about 750 μM, from about 25 μM to about 750 μM, from about 25 μM to about 700 μM, from about 25 μM to about 600 μM, from about 25 μM to about 500 μM, from about 25 μM to about 400 μM, from about 50 μM to about 150 μM, or from about 75 μM to about 125 μM). When calculating the amount of ABA to administer to a donor and/or recipient (e.g., a human transplantation donor or recipient), the volume of blood estimated to be present within the donor or recipient can be used to determine the amount of ABA to administer to have a total body ABA blood concentration. For example, about 77 mL of blood per kg of body weight can be used as an estimate for the volume of blood present within a human. In some cases, 5 mL of 100 mM ABA solution can be used to reach 100 μM ABA concentration in 5 L blood. In some cases, the U.S. Food and Drug guidelines for human dosing can be used to calculate an amount of ABA to be administered to a human based on the information provided herein (Guidance for Industry: Estimating the Maximum Safe Starting Dose in Initial Clinical Trials for Therapeutics in Adult Healthy Volunteers, U.S. Department of Health and Human Services, Food and Drug Administration, Center for Drug Evaluation and Research (CDER), July 2005; J:\!GUIDANC\5541fnlcln1.doc).

In some cases, ABA can be administered alone as a solution designed to have a particular concentration of ABA. For example, a solution of buffered saline can be formulated to have about 4 mg of ABA per mL. In that case, about 1 μL of the formulated solution can be administered per kg of body weight. In some cases, ABA can be formulated with other components. For example, ABA can be formulated together with heparin Na, microcirculation protectors, synthetic antioxidants, or combinations thereof to form a composition that can be administered to a transplantation donor or transplantation recipient to reduce the damage induced by the reperfusion of transplantation tissue.

In some cases, ABA can be chemically converted from its free base form to a pharmaceutically acceptable salt by reacting the free base with an equivalent amount of an acid or a base that forms a non-toxic salt. Such acids can be either inorganic or organic including, without limitation, hydrochloric acid, hydrobromic acid, fumaric acid, maleic acid, succinic acid, sulfuric acid, phosphoric acid, tartaric acid, acetic acid, citric acid, and oxalic acid. Suitable pharmaceutically acceptable base addition salts include, for example, metallic salts including alkali metal, alkaline earth metal, and transition metal salts such as, for example, calcium, magnesium, potassium, sodium, and zinc salts. Pharmaceutically acceptable base addition salts also can include organic salts made from basic amines such as, for example, N,N′-dibenzyl-ethylenediamine, chloroprocaine, choline, diethanolamine, ethylenediamine, meglumine (N-methylglucamine), and procaine. In some cases, ABA or a pharmaceutically acceptable salt thereof provided herein can be administered to a transplantation donor and/or transplantation recipient by itself or in combination with a carrier. Such carriers include, without limitation, sterile aqueous or non-aqueous solutions, suspensions, and emulsions. Examples of non-aqueous solvents include, without limitation, propylene glycol, polyethylene glycol, vegetable oils, and injectable organic esters. Aqueous carriers include, without limitation, water, alcohol, saline, and buffered solutions. In some cases, preservatives, flavorings, and other additives such as, for example, antimicrobials, anti-oxidants, chelating agents, inert gases, and the like can be present. It will be appreciated that ABA or a pharmaceutically acceptable salt thereof provided herein that is to be administered to a transplantation donor and/or transplantation recipient can contain zero, one, or more than one commonly known pharmaceutically acceptable carriers.

ABA or a composition that includes ABA as described herein can be administered to any part of a transplantation donor's or transplantation recipient's body. For example, ABA or a composition that includes ABA can be administered intravenously, intraarterially (e.g., via selective intra-arterial applications), orally, or intramuscularly. In some cases, the amount of ABA to be administered can be adjusted to account for a particular route of administration.

When directly exposing transplantation tissue that was removed from a donor to ABA prior to being transplanted into a recipient, any appropriate amount of ABA can be used. For example, the transplantation tissue can be exposed to a solution containing from about 1 μM to about 15 mM of ABA (e.g., from about 1 μM to about 10 mM, from about 1 μM to about 5 mM, from about 1 μM to about 1 mM, from about 1 μM to about 0.9 mM, from about 1 μM to about 0.8 mM, from about 1 μM to about 0.7 mM, from about 1 μM to about 0.6 mM, from about 1 μM to about 500 μM, from about 1 μM to about 400 μM, from about 1 μM to about 300 μM, from about 1 μM to about 200 μM, from about 1 μM to about 100 μM, from about 1 μM to about 50 μM, from about 1 μM to about 10 μM, from about 5 μM to about 10 mM, from about 10 μM to about 10 mM, from about 25 μM to about 10 mM, from about 50 μM to about 10 mM, from about 75 μM to about 10 mM, from about 100 μM to about 10 mM, from about 250 μM to about 10 mM, from about 500 μM to about 10 mM, from about 750 μM to about 10 mM, from about 1 mM to about 10 mM, from about 10 μM to about 1 mM, from about 20 μM to about 750 μM, from about 25 μM to about 750 μM, from about 25 μM to about 700 μM, from about 25 μM to about 600 μM, from about 25 μM to about 500 μM, from about 25 μM to about 400 μM, from about 50 μM to about 150 μM, or from about 75 μM to about 125 μM of ABA).

The invention will be further described in the following examples, which do not limit the scope of the invention described in the claims.

### EXAMPLES

### Example 1

### Treating Transplantation Recipients with ABA to Reduce Damage to Transplantation Tissue That is Induced by Reperfusion

Lewis inbred rats are randomly divided into five groups. The first group is a group of animals where one kidney organ is removed from each animal and replaced with a kidney from another animal within its group with no ABA treatment (operated untreated control). The other four groups are designed to have animals that undergo the same operation with the exception that ABA is administered intravenously to each animal 20 minutes before reperfusion of the transplanted kidney. ABA is administered at a final concentration of 10 μM for group 2, 50 μM for group 3, 100 μM for group 4, and 500 μM for group 5. Upon completion of the transplantation procedure, the animals are allowed to recover. After 10 to 30 days, the animals are anesthetized, and the transplanted kidneys are removed and evaluated.

### Example 2

### Treating Transplantation Donors with ABA to Reduce Damage to Transplantation Tissue That is Induced by Reperfusion Within Recipients

Lewis inbred rats are randomly divided into five groups. The first group is a group of animals where one kidney organ is removed from each animal and replaced with a kidney from another animal within its group with no ABA treatment (operated untreated control). The other four groups are designed to have recipient animals that receive a kidney transplantation from donors administered ABA intravenously 0.5 to 6 hours before removal of the kidneys. ABA is administered at a final concentration of 10 μM for group 2 donors, 50 μM for group 3 donors, 100 μM for group 4 donors, and 500 μM for group 5 donors. Upon completion of the transplantation procedure, the recipient animals are allowed to recover. After 10 to 30 days, the recipient animals are anesthetized, and the transplanted kidneys are removed and evaluated.

### Example 3

### Treating Transplantation Tissue Directly with ABA to Reduce Damage to the Transplantation Tissue That is Induced by Reperfusion Within Recipients

Lewis inbred rats are randomly divided into five groups. The first group is a group of animals where one kidney organ is removed from each animal and replaced with a kidney from another animal within its group with no ABA treatment (operated untreated control). The other four groups are designed to have recipient animals that receive a kidney that is directly exposed to ABA for 15 to 120 minutes before being transplanted into the recipient. ABA is used at a final concentration of 10 μM for group 2 transplantation tissue, 50 μM for group 3 transplantation tissue, 100 μM for group 4 transplantation tissue, and 500 μM for group 5 transplantation tissue. Upon completion of the transplantation procedure, the recipient animals are allowed to recover. After 10 to 30 days, the recipient animals are anesthetized, and the transplanted kidneys are removed and evaluated.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for reducing damage to transplantation tissue induced by reperfusion of said transplantation tissue in a mammalian recipient, wherein said method comprises administering a composition comprising abscisic acid to said mammalian recipient, wherein the level of damage of said transplantation tissue induced by reperfusion of said transplantation tissue in said mammalian recipient is reduced as compared to the level of damage of comparable transplantation tissue transplanted into a comparable mammalian recipient not administered said composition.

2. The method of claim 1, wherein said mammalian recipient is a human.

3. (canceled)

4. The method of claim 1, wherein said composition is administered to said mammalian recipient during a transplantation procedure.

5. (canceled)

6. The method of claim 1, wherein said composition comprises between 5 and 95 percent by weight said abscisic acid.

7-9. (canceled)

10. The method of claim 1, wherein said administration comprises an intravenous administration.

11. A method for reducing damage to transplantation tissue induced by reperfusion of said transplantation tissue in a mammalian recipient, wherein said method comprises administering a composition comprising abscisic acid to a donor of said transplantation tissue, wherein the level of damage of said transplantation tissue induced by reperfusion of said transplantation tissue in said mammalian recipient is reduced as compared to the level of damage of comparable transplantation tissue obtained from a comparable donor not treated with said composition and implanted into a comparable mammalian recipient.

12. The method of claim 11, wherein said mammalian recipient is a human.

13. The method of claim 11, wherein said donor is a human.

14-15. (canceled)

16. The method of claim 11, wherein a second composition comprising abscisic acid is administered to said mammalian recipient.

17. (canceled)

18. The method of claim 11, wherein said composition comprises between 5 and 95 percent by weight said abscisic acid.

19-21. (canceled)

22. The method of claim 16, wherein said second composition comprises between 5 and 95 percent by weight said abscisic acid.

23-25. (canceled)

26. The method of claim 11, wherein said administration of said composition comprises an intravenous administration.

27. The method of claim 16, wherein said administration of said second composition comprises an intravenous administration.

28. A method for reducing damage to transplantation tissue induced by reperfusion of said transplantation tissue in a mammalian recipient, wherein said method comprises:
(a) contacting transplantation tissue with a composition comprising abscisic acid prior to implanting said transplantation tissue into said mammalian recipient,
(b) implanting said transplantation tissue into said mammalian recipient, and
(c) reperfusing said transplantation tissue,
wherein the level of damage of said transplantation tissue induced by reperfusion of said transplantation tissue in said mammalian recipient is reduced as compared to the level of damage of comparable transplantation tissue not contacted with said composition and transplanted into a comparable mammalian recipient.

29. The method of claim 28, wherein said mammalian recipient is a human.

30-31. (canceled)

32. The method of claim 28, wherein said composition comprises between 5 and 95 percent by weight said abscisic acid.

33-35. (canceled)

36. The method of claim 28, wherein said method comprises administering a composition comprising abscisic acid to said mammalian recipient.

37. (canceled)

38. The method of claim 28, wherein said method comprises administering a composition comprising abscisic acid to a donor of said transplantation tissue prior to removal of said transplantation tissue from said donor.

39. The method of claim 28, wherein said method comprises administering a composition comprising abscisic acid to a donor of said transplantation tissue at least 10 minutes prior to removal of said transplantation tissue from said donor.

40. (canceled)
